(19) United States
(12) Patent Application Publication
(10) Pub. No.: US 2025/0209411 A1
(43) Pub. Date: Jun. 26, 2025

(54) MEDICATION MANAGEMENT SYSTEM

(71) Applicants: DENSO WAVE INCORPORATED, Chita-gun, Aichi-pref. (JP); MEDIOTOLOGY INC., Tokyo (JP)

(72) Inventors: Yousuke OKAYAMA, Chita-gun (JP); Koji KONOSU, Chita-gun (JP); Shoji SATO, Chita-gun (JP); Takehiro KIMURA, Tokyo (JP)

(73) Assignees: DENSO WAVE INCORPORATED, Chita-gun, Aichi-pref. (JP); MEDIOTOLOGY INC., Tokyo (JP)

(21) Appl. No.: 18/847,526

(22) PCT Filed: Mar. 9, 2023
(86) PCT No.: PCT/JP2023/009079

§ 371 Date: Sep. 16, 2024

(30) Foreign Application Priority Data
Mar. 16, 2022 (JP) 2022-041371

(51) Int. Cl. G16Y 10/60; G06Q 10/083; G16Y 40/10

(52) U.S. Cl. CPC G06Q 10/083; G16Y 10/60; G16Y 40/10

(57) ABSTRACT

A medication management system including: a cloud server; an inner box containing medications, a wireless tag in which a box ID is recorded being attached to the inner box; a reading unit reading the box ID from the wireless tag of the inner box; and a communication unit transmitting information including the read box ID to the cloud server; wherein the cloud server is configured to: receive the box ID and delivery time information related to a scheduled time when the inner box is to be delivered to the patient; store the box ID, the delivery time information, and patient information related to the patient; determine whether the box ID associated with the delivery time information has been received from the communication unit before a predetermined time has elapsed since scheduled time indicated by the delivery time information; and give notification of information that is based on a determination result.

### TECHNICAL FIELD

The present disclosure relates to a medication management system.

### BACKGROUND ART

Conventionally, when a patient sees a doctor at a hospital to get a prescription issued from the doctor, a medication dispensed based on the prescription is issued to the patient at a pharmacy or the like. Currently, in order to prevent spread of infectious diseases or the like, a medication prescribed based on a past prescription is issued by delivery without requiring a patient to come to a pharmacy or the like.

As a technique related to such delivery, for example, there is known a gift shipping system disclosed in PTL 1 below. In this gift shipping system, a procedure for purchasing a product is performed via the internet using purchaser information registered in advance, so that the product is shipped to a specified shipping destination. Such a shipping system allows a purchaser to purchase a product without entering purchaser information every time he/she purchases a product.

### CITATION LIST

### Patent Literature

PTL 1: JP 4937434 B

### SUMMARY OF THE INVENTION

It is desirable to take a medication at a predetermined time. However, there is a possibility that a medication for a patient to take may not be delivered by the time to take the medication through medication delivery.

The present disclosure has been made to solve the above problem, and an object thereof is to provide a medication management system that can give notification of whether a medication for a patient to take has been delivered by the time to take the medication.

A medication management system according to an embodiment of the present disclosure is a medication management system including:
a cloud server;
an inner box configured to contain one or more doses of medication for a patient to take, wherein a wireless tag in which a box identifier, ID, is recorded is attached to the inner box;
a container unit configured to contain the inner box;
a reading unit configured to read the box ID from the wireless tag of the inner box contained in the container unit; and
a first communication unit configured to acquire the box ID read by the reading unit and to transmit information including the box ID to the cloud server;
wherein the cloud server includes:
a second communication unit configured to receive, from an apparatus of a delivery origin, the box ID and delivery time information related to a scheduled time when the inner box is to be delivered to the patient;
a storage unit configured to store the box ID, the delivery time information, and patient information related to the patient, wherein the box ID is associated with the patient information and the delivery time information is associated with the box ID;
a determination unit configured to determine whether the box ID associated with the delivery time information has been received from the first communication unit before a predetermined time has elapsed since scheduled time indicated by the delivery time information; and
a notification unit configured to give notification of information that is based on a determination result of the determination unit.

With this configuration, if the inner box has not reached the patient, the reading unit does not read the box ID, and thus the first communication unit does not transmit the box ID to the cloud server. That is, the second communication unit of the cloud server does not receive the box ID from the first communication unit. The determination unit of the cloud server determines, when the predetermined time has elapsed since the scheduled time indicated by the delivery time information, that the inner box has not reached the patient. This determination is also referred to as abnormality determination. In a case of the abnormality determination, the notification unit gives notification of information that is based on the abnormality determination. In this manner, the medication management system according to embodiments of the present disclosure can give notification of whether a medication for a patient to take has been delivered by the time to take the medication.

### BRIEF DESCRIPTION OF DRAWINGS

FIG. 1 is an explanatory diagram illustrating a schematic configuration of a medication management system according to a first embodiment of the present disclosure.

FIG. 2A is a perspective view illustrating an inner box according to the first embodiment of the present disclosure.

FIG. 2B is an explanatory diagram for explaining a state where the inner box is packaged in a cover or the like according to the first embodiment of the present disclosure.

FIG. 3 is a block diagram schematically illustrating a functional configuration of an information processing apparatus according to the first embodiment of the present disclosure.

FIG. 4 is a block diagram schematically illustrating a functional configuration of a cloud server according to the first embodiment of the present disclosure.

FIG. 5 is an explanatory diagram for explaining patient information, box IDs, delivery date and time information, and reception date and time information that are registered in a database of the cloud server according to the first embodiment of the present disclosure.

FIG. 6 is an explanatory diagram for explaining a medication to which a medication tag is attached according to a second embodiment of the present disclosure.

FIG. 7 is a flowchart illustrating a flow of medication status management processing performed by a control unit of the cloud server according to the second embodiment of the present disclosure.

FIG. 8A is an explanatory diagram for explaining initial information registered in a database of the cloud server according to the second embodiment of the present disclosure.

FIG. 8B is an explanatory diagram for explaining updating of the information registered in the database of the cloud server according to the second embodiment of the present disclosure.

FIG. 9 is an explanatory diagram for explaining information registered in the database of the cloud server according to a modification example of the second embodiment of the present disclosure.

FIG. 10 is an explanatory diagram for explaining an example where a display terminal acts as a relay between the information processing apparatus of a container unit and the cloud server in a medication management system according to a modification example of the second embodiment of the present disclosure.

FIG. 11 is a block diagram illustrating a hardware configuration of the information processing apparatus according to embodiments of the present disclosure.

FIG. 12 is a block diagram illustrating a hardware configuration of the cloud server according to embodiments of the present disclosure.

### DESCRIPTION OF EMBODIMENTS

If a medication is not properly taken, its effect may not be as expected. It is necessary for a patient to take an appropriate dose of a prescribed type of medication within a predetermined time window. However, in a case where a medication is not directly passed to a patient at a pharmacy but is delivered, a shipping person of the pharmacy that is a delivery origin is unable to confirm whether the medication for the patient has reached the patient. Thus, the shipping person is unable to know a situation where a part or all of the medication has not reached on time (i.e., delayed delivery) and a situation where the medication has been delivered to a wrong delivery destination and a situation where a wrong medication has been delivered (i.e., incorrect delivery), making it difficult to promptly take an action for these situations (i.e., nonarrival of medication). If such nonarrival of medication occurs, the patient may fail to take a medication within a predetermined time window due to a shortage in the medication the patient should take, or a patient who has received a medication that is not prescribed for the patient may take the medication.

According to embodiments of the present disclosure, there is provided a medication management system that can promptly determine whether a medication for a patient to take has been correctly delivered to the patient. In other words, there is provided a medication management system that can give notification of whether a medication for a patient to take has been delivered by the time to take the medication.

### First Embodiment

Hereinafter, a medication management system (or stated differently, medication delivery management system) according to a first embodiment of the present disclosure will be described with reference to the drawings. A medication management system 1 according to this embodiment manages, for each patient, one or more types of medications delivered based on a past prescription. Specifically, in the medication management system 1, an inner box 20 that contains a plurality of medications 10 which a patient should take is prepared according to a prescription at a pharmacy or the like that is a delivery origin. The inner box 20 is delivered to a home or the like of the patient and is stored in a container unit 30 that is installed in advance in the home of the patient. The inner box 20 is stored in the container unit 30 by a person (e.g., the patient) who is at the home of the patient. Then, in the medication management system 1, a cloud server 60 manages information read from a wireless tag attached to the inner box 20 containing the plurality of medications 10, thereby managing information related to the plurality of medications 10 delivered to the patient. For the sake of convenience, a packaged medication is also simply described as a medication 10.

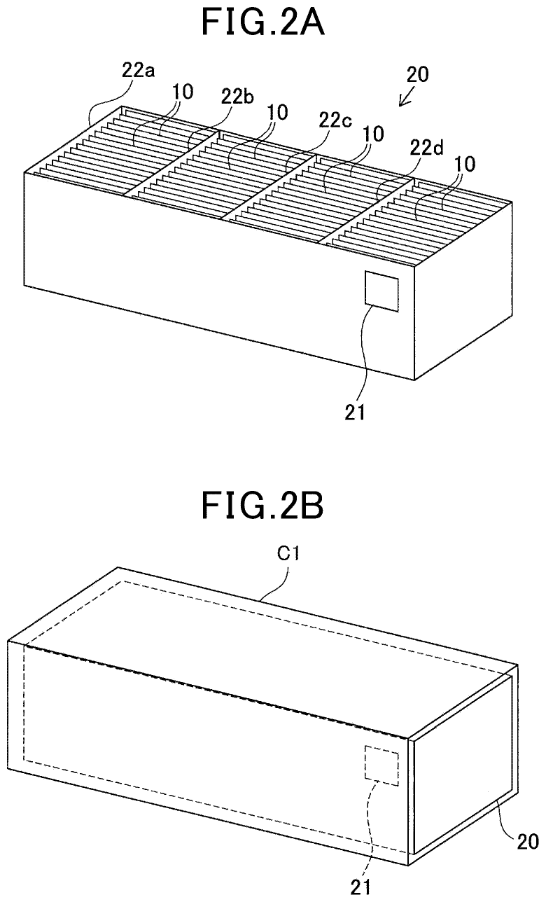

As illustrated in FIG. 2A, the inner box 20 has a plurality of compartments 22a to 22d, each of which contains a plurality of medications 10 which the patient should take in a predetermined orientation. As illustrated in FIG. 2B, when being packaged in a cover C1 or the like, the inner box 20 functions as a packaging box of the plurality of medications 10 for delivery from the pharmacy to the home of the patient. The inside of the inner box 20 is partitioned into the compartments 22a to 22d, which are container spaces corresponding to types of the plurality of medications 10. A wireless tag (hereinafter, also referred to as a box tag 21) such as a radio frequency (RF) tag in which a box ID for distinguishing the inner box 20 from other inner boxes 20 is recorded is attached to the inner box 20.

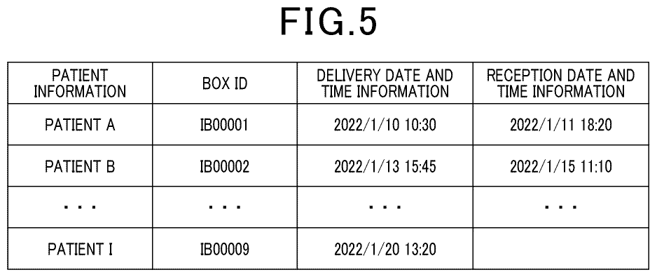

A format of the box ID is a predetermined format in this embodiment. Thus, box IDs can be discriminated from information recorded in another wireless tag that is different from a box tag 21. Specifically, as illustrated in FIG. 5 described later, the predetermined format begins with, for example, ‘IB’.

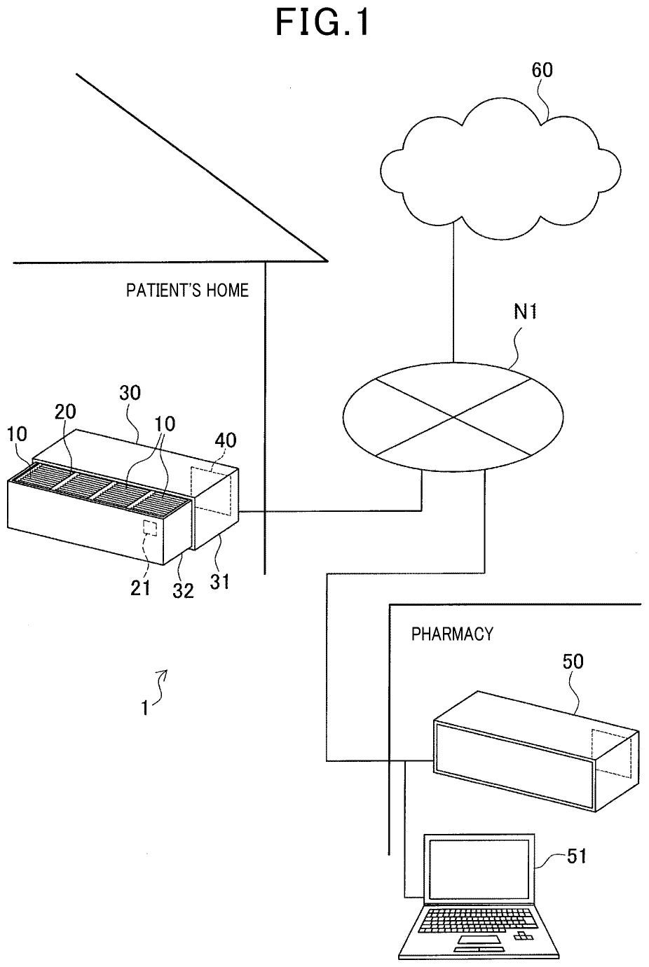

The container unit 30 contains an inner box 20. For example, the container unit 30 functions as a medicine cabinet. As illustrated in FIG. 1, the container unit 30 includes an open-front main unit 31 of the container unit 30 and a drawer unit 32 that can be pulled out and put in through the opening. The main unit 31 is formed such that, in a state where there is the drawer unit 32 containing an inner box 20 put into the main unit 31 through the opening (hereinafter, also referred to as a set state), the main unit 31 and the front surface of the drawer unit 32 together form a substantially box shape. The upper side of the drawer unit 32 is open in a rectangular shape to fit the outline of an inner box 20 so that an inner box 20 can be stored therein from above.

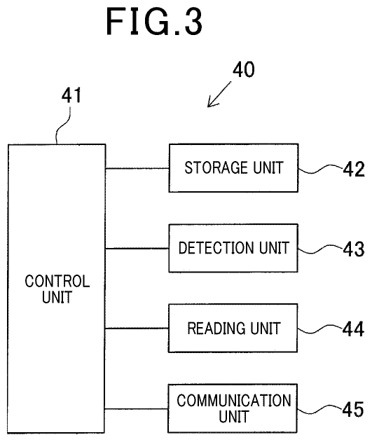

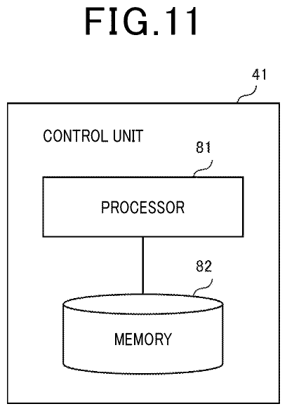

An information processing apparatus 40 is embedded in the container unit 30. The information processing apparatus 40 transmits a box ID read from a box tag 21 to the cloud server 60 when the set state is detected. As illustrated in FIG. 3, the information processing apparatus 40 includes a control unit 41, a storage unit 42, a detection unit 43, a reading unit 44, and a communication unit 45. The storage unit 42 stores information related to a patient (hereinafter, also referred to as patient information) in advance. As illustrated in FIG. 11, the control unit 41 may be implemented by, for example, a processor 81 such as a central processing unit (CPU) or a micro processing unit (MPU) and a memory 82 such as a random-access memory (RAM) or a read-only memory (ROM). The storage unit 42 may be implemented by, for example, a semiconductor memory.

The detection unit 43 is a contact sensor or the like for detecting the set state. The detection unit 43 is provided deep inside the main unit 31. In the set state, the detection unit 43 detects contact with the drawer unit 32 and outputs a predetermined detection signal to the control unit 41. Note that the detection unit 43 is not limited to the contact sensor and may be another sensor capable of detecting the set state.

The reading unit 44 reads information from specific wireless tags including a box tag 21. The reading unit 44 communicates with the specific wireless tags using electromagnetic waves via an antenna (not illustrated) under control of the control unit 41. In this way, the reading unit 44 reads and writes data stored in the specific wireless tags.

The communication unit 45 communicates with external equipment. For example, the communication unit 45 communicates with other external equipment such as the cloud server 60 via a network N1 (e.g., the internet or the like) under control of the control unit 41.

In the information processing apparatus 40, when the detection unit 43 detects the set state, the control unit 41 starts read information transmission processing. In the read information transmission processing, in a case where information read by the reading unit 44 immediately after the detection of the set state is a box ID in the predetermined format, the box ID is transmitted to the cloud server 60 via the communication unit 45 along with the patient information stored in the storage unit 42.

Thus, for example, when the patient stores an inner box 20 received from the pharmacy in the container unit 30, the information processing apparatus 40 transmits a box ID read from a box tag 21 of the stored inner box 20 to the cloud server 60 along with the patient information. On the other hand, even when a different object other than an inner box 20 is contained in the container unit 30 and such another object has a wireless tag attached thereto, unless information read from the wireless tag is in the predetermined format, the read information is not transmitted to the cloud server 60. Note that, in this embodiment, the information processing apparatus 40 operates with power supplied from a battery not illustrated. However, the information processing apparatus 40 is not limited thereto, and may operate with power supplied from, for example, a commercial power source.

In the pharmacy that is the delivery origin of a plurality of medications 10, a container apparatus 50 for delivery origins having functions similar to those of the container unit 30 and the information processing apparatus 40 is installed. An inner box 20 that contains a plurality of medications 10 to be delivered to the patient is prepared. When the inner box 20 is stored in the container apparatus 50, a box ID read from a box tag 21 of the inner box 20 is transmitted to the cloud server 60 along with medication information related to the plurality of the medications 10 contained in the inner box 20 and patient information. Note that the container apparatus 50 may acquire the medication information and the patient information to be transmitted by, for example, reading them from a separately prepared wireless tag or using another acquisition means.

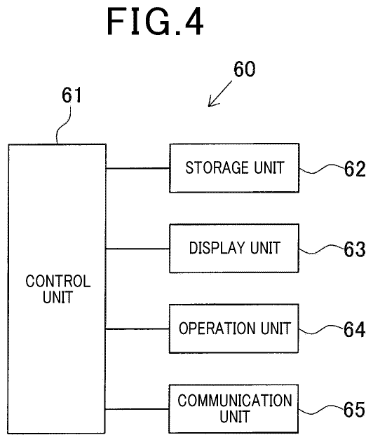

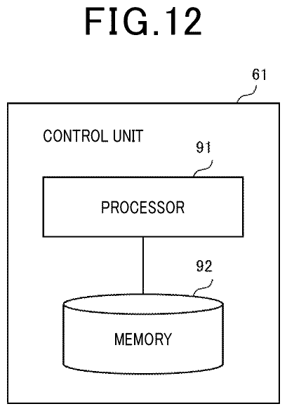

The cloud server 60 is an information processing apparatus in which patient information related to a patient and medication information related to a plurality of medications 10 for the patient to take are stored. The medication information is associated with the patient information, so that a database including the patient information and the medication information is constructed. As illustrated in FIG. 4, the cloud server 60 includes a control unit 61, a storage unit 62, a display unit 63, an operation unit 64, and a communication unit 65. As illustrated in FIG. 12, the control unit 61 may be implemented by, for example, a processor 91 such as a CPU and a memory 92 such as a RAM or a ROM. The storage unit 62 may be implemented by, for example, a storage such as a semiconductor memory or a hard disk drive (HDD).

When the inner box 20 prepared in the pharmacy as described above is delivered to the patient, the cloud server 60 receives the box ID, the patient information, and the medication information from the container apparatus 50 of the pharmacy via the communication unit 65. Date and time when the information is received from the container apparatus 50 is associated with the box ID and the patient information as delivery date and time information. The delivery date and time information is registered in the database constructed in the storage unit 62. For example, in a case where the patient information ‘Patient I’ and the box ID ‘IB00009’ are received from the container apparatus 50 on the date and time ‘2022 Jan. 20 13:20’, as illustrated in FIG. 5, the date and time of receipt is registered as delivery date and time information in the database along with the box ID. Note that delivery date and time information is not limited to the information of date and time when a box ID and patient information are received, and may be, for example, date and time information separately received from the pharmacy. For the sake of convenience, medication information is omitted in FIG. 5.

Then, in the cloud server 60, date and time when a box ID and patient information are received from the information processing apparatus 40 is associated with the box ID and the patient information as reception date and time information. The reception date and time information is registered and stored in the database. For example, in a case where the patient information ‘Patient B’ and the box ID ‘IB00002’ are received from the information processing apparatus 40 on the date and time ‘2022 Jan. 15 11:10’, as illustrated in FIG. 5, the date and time of receipt is registered as reception date and time information. The reception date and time information is associated with the box ID that has been already registered.

In the cloud server 60, the control unit 61 performs processing to determine whether an inner box 20 has been correctly delivered to a patient using information stored in the storage unit 62 (hereinafter, also referred to as delivery correctness determination processing). Specifically, the control unit 61 determines whether a box ID associated with delivery time information has been received from the communication unit 45 of the information processing apparatus 40 before a predetermined time has elapsed since scheduled time indicated by the delivery time information. More specifically, the control unit 61 determines whether the time difference between the delivery date and time information and the reception date and time information does not exceed a predetermined time. If the time difference does not exceed the predetermined time, it is determined that the inner box 20 has been correctly delivered to the patient. For example, as illustrated in FIG. 5, for the box ID ‘IB00002’, the time difference between the delivery date and time information ‘2022 Jan. 13 15:45’ and the reception date and time information ‘2022 Jan. 15 11:10’ does not exceed the predetermined time (e.g., 72 hours), and thus it is determined that the inner box 20 has been correctly delivered to the patient B associated with the box ID.

On the other hand, in a case where nonarrival of medication as described above occurs, the box ID associated with the delivery date and time information may not be received from the information processing apparatus 40 even after the predetermined time has elapsed since the delivery date and time information. In this case, in the delivery correctness determination processing, it is determined that the inner box 20 has not been correctly delivered to the patient. That is, an abnormality determination is made. For example, as illustrated in FIG. 5, for the box ID ‘IB00009’, if the box ID ‘IB00009’ is not received from the information processing apparatus 40 even when the predetermined time has elapsed since the delivery date and time information ‘2022 Jan. 20 13:20’, it is determined that the inner box 20 has not been correctly delivered to the patient.

Furthermore, in the delivery correctness determination processing, the control unit 61 determines whether an inner box 20 has been correctly delivered to a patient based on whether patient information and a box ID that are received from the information processing apparatus 40 are associated with each other in the storage unit 62. In other words, the control unit 61 determines whether an inner box 20 has been correctly delivered to a patient based on patient information and a box ID that are received from the information processing apparatus 40 and patient information and a box ID that are stored in the storage unit 62. For example, in the delivery correctness determination processing, if the received box ID does not match any box ID associated with the received patient information in the storage unit 62, it is determined that the inner box 20 has not been correctly delivered to the patient. In this case, the inner box 20, which should be delivered to a different patient other than the patient who has received the inner box, has been delivered to the patient by mistake.

Upon the abnormality determination as described above, the control unit 61 performs abnormality determination notification processing. In the abnormality determination notification processing, notification information indicating that an inner box 20 has not been correctly delivered to a patient is transmitted to a predetermined terminal 51 installed in a pharmacy that is the delivery origin of the inner box 20 via the communication unit 65 along with the patient information and the box ID. Note that the control unit 61 that performs the delivery correctness determination processing may correspond to a “determination unit” that determines whether an inner box 20 has been correctly delivered to a patient. The control unit 61 and the communication unit 65 that perform the abnormality determination notification processing may correspond to a “notification unit” that gives notification of a determination result of the “determination unit”.

The predetermined terminal 51 installed in the pharmacy displays the received notification information on a screen. This allows, for example, a pharmacist to easily know the patient that is a delivery destination and the delivery date and time of the inner box 20 that has not been correctly delivered. In this case, for example, the pharmacist checks a delivery status with the patient, a delivery company, or the like and, if necessary, asks the delivery company to deliver a new inner box 20. This promptly allows the inner box 20 (i.e., medications 10) to be redelivered.

In a case where the box ID has not been received before the predetermined time has elapsed since the delivery date and time, prior to transmitting the notification information to the delivery origin (e.g., pharmacy), the cloud server 60 may notify a terminal owned by the patient of information prompting the patient to store the inner box 20 in the container unit 30. In a case where the box ID is not received from the container unit 30 at the patient's home even when a predetermined period has elapsed since the notification, the cloud server 60 may transmit the notification information to the delivery origin. In addition, in a case where it is determined that the inner box 20 has been correctly delivered to the patient, the cloud server 60 may transmit the determination result to the delivery origin of the inner box 20.

As described above, in the medication management system 1 according to this embodiment, an inner box 20 contains a plurality of medications 10 for a patient to take, and a box tag 21 in which a specific box ID is recorded is attached to the inner box 20, which is stored in the container unit 30 by the patient. In a state where the inner box 20 is contained in the container unit 30, the box ID is read from the box tag 21 by the reading unit 44, and information including the box ID is transmitted to the cloud server 60 via the communication unit 45. In the cloud server 60, when an inner box 20 is delivered to a patient, a box ID corresponding to the inner box 20 and delivery date and time information that are received from the container apparatus 50 of the delivery origin are stored in the storage unit 62 in association with patient information and medication information. Then, if the box ID stored in association with the delivery date and time information in the storage unit 62 is not received even when a predetermined time has elapsed since the delivery date and time of the delivery date and time information, it is determined that the inner box 20 has not been correctly delivered to the patient.

In other words, the medication management system according to this embodiment is a medication management system including: a cloud server; an inner box configured to contain one or more medications for a patient to take, wherein a wireless tag in which a box ID is recorded is attached to the inner box; a container unit configured to contain the inner box; a reading unit configured to read the box ID from the wireless tag of the inner box contained in the container unit; and a first communication unit configured to acquire the box ID read by the reading unit and to transmit information including the box ID to the cloud server; wherein the cloud server includes: a second communication unit configured to receive, from an apparatus of a delivery origin, the box ID and delivery time information related to a scheduled time when the inner box is to be delivered to the patient; a storage unit configured to store the box ID, the delivery time information, and patient information related to the patient, wherein the box ID is associated with the patient information and the delivery time information is associated with the box ID; a determination unit configured to determine whether the box ID associated with the delivery time information has been received from the first communication unit before a predetermined time has elapsed since scheduled time indicated by the delivery time information; and a notification unit configured to give notification of information that is based on a determination result of the determination unit.

With this configuration, if the inner box 20 has not reached the patient, the reading unit 44 does not read the box ID, and thus the cloud server 60 does not receive the box ID. Therefore, even when the predetermined time has elapsed since the delivery date and time information, the cloud server 60 does not receive the associated box ID, and thus can determine that the inner box 20 has not been correctly delivered to the patient. In a case of an abnormality determination, the delivery origin which has received notification according to the determination result can check a delivery status with the patient and a delivery company and, if necessary, arrange a new inner box 20 to be delivered. In other words, the medication management system 1 can give notification of whether the medication 10 for the patient to take has been delivered by the time to take the medication. In this manner, whether a medication 10 for a patient to take has been correctly delivered can be promptly determined, thereby making it possible to promptly take an action for nonarrival of medication 10 when it occurs. Note that the information related to a result of an abnormality determination as described above may be transmitted to a display terminal 70 described later so that a patient who manages the display terminal 70 is notified of the information.

In addition, the information processing apparatus 40 transmits information including a box ID and patient information to the cloud server 60. In the delivery correctness determination processing performed by the control unit 61 of the cloud server 60, it is determined whether the inner box 20 has been correctly delivered to the patient based on whether the patient information and the box ID that are received from the information processing apparatus 40 are associated with each other in the storage unit 62.

In other words, the first communication unit is configured to transmit information including the box ID and the patient information to the cloud server, and the determination unit is further configured to determine whether the box ID received from the first communication unit matches the box ID that is associated, in the storage unit, with the patient information received from the first communication unit.

With this configuration, for example, if an inner box 20 to be delivered to a certain patient has been delivered to another patient by mistake, the patient information received by the cloud server 60 is different from the patient information stored in the storage unit 62 in association with the box ID received at the same timing. In this manner, if these pieces of patient information do not match, it can be determined that the inner box 20 has not been correctly delivered to the patient. When the container unit 30 contains an incorrect inner box 20, notification of a result of an abnormality determination is immediately given. Thus, a delivery origin which has received the notification of the result of the abnormality determination can inform the patient not to take the medication 10 contained in the inner box 20, thereby preventing the patient from taking a wrong medication. Note that the cloud server 60 may transmit information related to the result of the abnormality determination to a display terminal 70 described later. This allows the patient to be directly notified not to take the medication 10 contained in the inner box 20.

In addition, a box ID is generated in a predetermined format to indicate that it is information recorded in a box tag 21 attached to an inner box 20. If the reading unit 44 has read only information other than a box ID generated in the predetermined format, the information processing apparatus 40 does not transmit such other information to the cloud server 60.

In other words, a format of the box ID is a predetermined format corresponding to information recorded in the wireless tag attached to the inner box, and the first communication unit is configured not to transmit information that has been read by the reading unit but is not in the predetermined format to the cloud server.

With this configuration, when the container unit 30 contains an inner box 20, information such as the box ID of the inner box 20 is transmitted to the cloud server 60. On the other hand, when the container unit 30 contains another object to which a wireless tag is attached, information read from the wireless tag is not transmitted to the cloud server 60. This can prevent the cloud server 60 from receiving unnecessary information other than information of an inner box 20.

### Second Embodiment

Next, a medication management system according to a second embodiment will be described with reference to the drawings. This embodiment is different from the first embodiment mainly in that a wireless tag in which a medication ID is recorded is attached to a medication 10. Note that components substantially the same as those in the first embodiment are denoted by the same reference signs, and descriptions thereof are omitted.

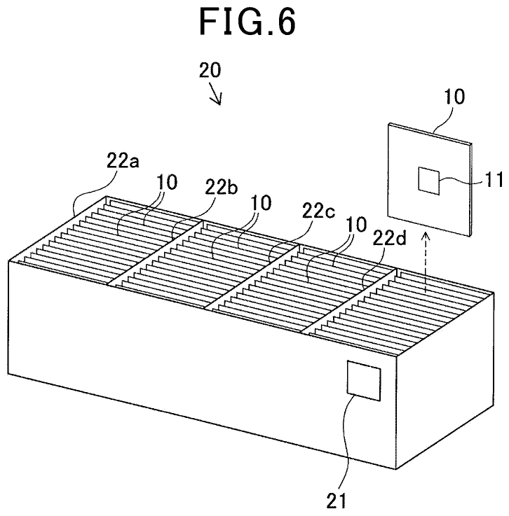

In this embodiment, in the cloud server 60, when it is determined that an inner box 20 has been correctly delivered to a patient, processing for managing medication statuses, such as a remaining amount, of a plurality of medications 10 contained in the inner box 20 is performed. As illustrated in FIG. 6, a wireless tag (hereinafter, also referred to as a medication tag 11) in which a specific medication ID such as a serial number is recorded is attached to each of the medications 10 contained in the inner box 20. In this embodiment, in order to protect privacy of a patient, a unique random value is adopted as a medication ID, by which alone a type of medication 10 is not known. In another embodiment, a format of a medication ID may be such a predetermined format that medication information is identified from at least a part of the medication ID. This makes it possible to identify medication information related to a medication 10 from a medication ID recorded in a medication tag 11 attached to the medication 10.

In a pharmacy that is a delivery origin, by storing an inner box 20 in the container apparatus 50, a box ID read from a box tag 21 of the inner box 20 and a plurality of medication IDs respectively read from a plurality of medication tags 11 are transmitted to the cloud server 60 along with patient information and the like.

In read information transmission processing performed by the information processing apparatus 40 according to this embodiment, a box ID and a plurality of medication IDs are read and information including the box ID and the plurality of medication IDs that have been read is transmitted to the cloud server 60. In this embodiment, at a timing when the set state is detected, a box ID and a plurality of medication IDs are read. Thus, not only the first time an inner box 20 is stored in the container unit 30 but every time the inner box 20 is placed in the container unit 30, a box ID and a plurality of medication IDs are read. For example, when a patient has pulled out the drawer unit 32 from the main unit 31 to pick up a dose of medication 10 which the patient should take from an inner box 20 and then pushed back the drawer unit 32, a box ID and a plurality of medication IDs are read and the box ID and the plurality of medication IDs that have been read are transmitted to the cloud server 60 along with patient information. That is, every time the patient picks up a dose of the medication 10, medication IDs of remaining medications 10 are transmitted to the cloud server 60.

The control unit 61 of the cloud server 60 uses a box ID and a plurality of medication IDs that are received from the information processing apparatus 40 to perform medication status management processing for managing medication statuses including a remaining amount of medication 10 for each inner box 20. The storage unit 62 of the cloud server 60 stores, as delivery information, a box ID and a plurality of medication IDs that have been received from the container apparatus 50 installed in the delivery origin. The plurality of medication IDs are associated with at least the box ID. Based on the delivery information, the number of medications 10 to be delivered can be obtained. In addition, the storage unit 62 of the cloud server 60 stores, for a medication ID of each of medications 10 under management, information related to a type of the medication 10, medication time, a dosage, and the like that are identified from the medication ID. That is, information related to medication 10 is associated with at least a medication ID. For example, when an inner box 20 is delivered from the pharmacy, a pharmacist inputs information related to medication 10 for a medication ID. The medication ID and the input information related to the medication 10 are transmitted from the container apparatus 50 to the cloud server 60 and are registered in a database in the storage unit 62 in association with each other.

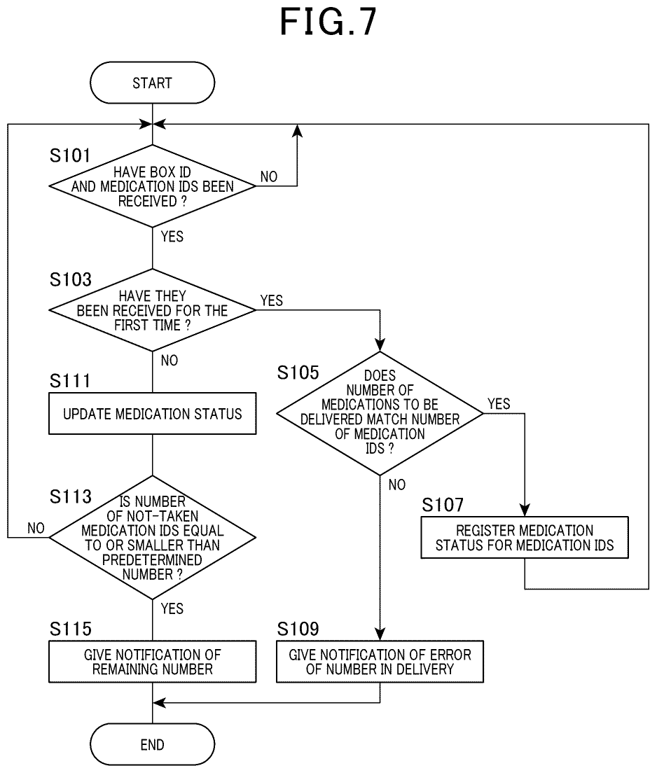

The medication status management processing performed by the control unit 61 will be described in detail below with reference to the flowchart illustrated in FIG. 7. Upon starting the medication status management processing, in step S101 of FIG. 7, the control unit 61 determines whether a box ID and a plurality of medication IDs have been received via the communication unit 65. If a box ID and a plurality of medication IDs have been received from the information processing apparatus 40 (YES in S101), in step S103, the control unit 61 determines whether the box ID and the plurality of medication IDs have been received for the first time.

If the box ID and the plurality of medication IDs are received when a patient stores and sets an inner box 20 in the container unit 30 for the first time (YES in S103), in step S105, the control unit 61 determines whether the number of medications 10 to be delivered that is obtained from delivery information that is associated with the received box ID and is stored in the storage unit 62 matches the number of the received medication IDs.

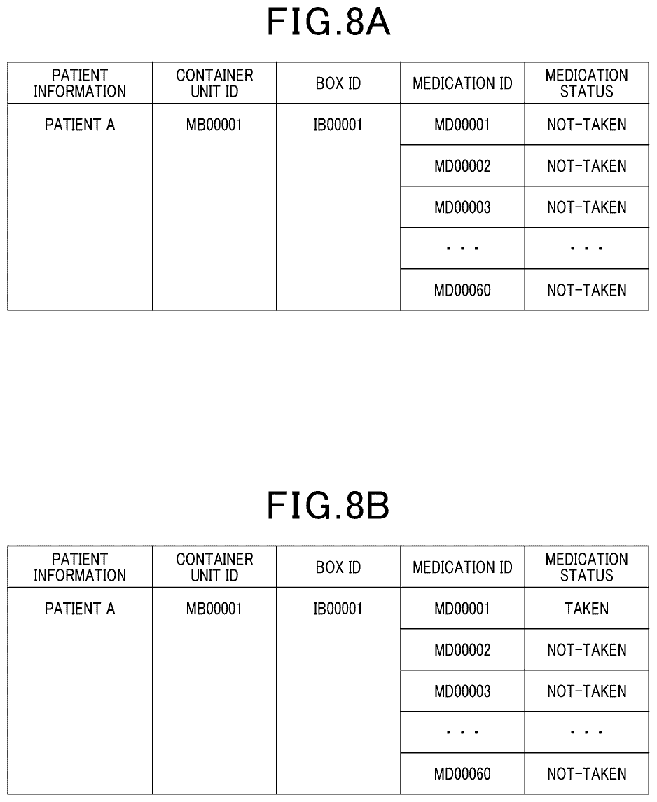

If the number of medications 10 to be delivered matches the number of the received medication IDs (YES in S105), the determination result means that the inner box 20 has been correctly delivered without any loss of the medications 10. In this case, in step S107, a medication status is set in association with a combination of the box ID and each of the plurality of medication IDs that have been received. The medication status is registered in a database. Note that the medication status may be associated with each medication ID. For example, as illustrated in FIG. 8A, assume a case where the patient information ‘Patient A’, the box ID ‘IB00001’, and the sixty medication IDs ‘MD00001’ to ‘MD00060’ are received from the information processing apparatus 40 with the container unit ID ‘MB00001’. In this case, when the number of the medications 10 to be delivered that is stored as the delivery information is sixty, a medication status is set in association with a combination of the box ID and each of the sixty medication IDs. At this time, the medication statuses for all of the sixty medication IDs are set to ‘Not-Taken’. In step S107, date and time when the box ID and the plurality of medication IDs are received is registered as reception date and time information. Subsequently, the processing returns to step S101 described above.

Meanwhile, if the number of medications 10 to be delivered does not match the number of the received medication IDs (NO in S105), the determination result means that a part of the medication 10 has been lost during delivery. In this case, in step S109, notification information indicating the occurrence of loss is transmitted to the delivery origin of the inner box 20 via the communication unit 65 along with patient information and the box ID. In other words, the delivery origin is notified of an error of the number in delivery.

The notification information received by a predetermined terminal 51 installed in the delivery origin is displayed on a screen. This allows a pharmacist of the delivery origin to easily know the patient to which the inner box 20 experiencing the loss is delivered and the delivery date and time. Therefore, the pharmacist can promptly take an action for the nonarrival of the medication 10 by checking a delivery status with the patient and a delivery company and arranging a new inner box 20 to be delivered.

If the box ID and a plurality of medication IDs have been previously received (NO in S103), in step S111, the control unit 61 considers a medication 10 of which medication ID has not been received among the plurality of registered medication IDs to have been taken by the patient and updates the medication status of that medication ID to Taken. For example, in a case where initial registration is as illustrated in FIG. 8A, assume that the patient takes a dose of the medication of the medication ID ‘MD00001’. At this time, the patient information ‘Patient A’, the container unit ID ‘MB00001’, the box ID ‘IB00001’, and the fifty-nine medication IDs ‘MD00002’ to ‘MD00060’ are received. As illustrated in FIG. 8B, the control unit 61 considers the dose of the medication 10 of the medication ID ‘MD00001’ to have been taken by the patient and updates the medication status of that medication ID to ‘Taken’. Meanwhile, the medication statuses of the medication IDs ‘MD00002’ to ‘MD00060’ are kept to be ‘Not-Taken’.

Subsequently, in step S113, the control unit 61 determines whether the number of Not-Taken medication IDs is equal to or smaller than a predetermined number. If the number of the Not-Taken medication IDs is larger than the predetermined number (NO in S113), a remaining number of the medications 10 in the inner box 20 is considered to be sufficient and the processing returns to step S101 described above.

Meanwhile, if the number of the Not-Taken medication IDs is equal to or smaller than the predetermined number (YES in S113), the control unit 61 considers the number of the medications 10 in the inner box 20 to be insufficient and, in step S115, notifies the delivery origin of information related to the remaining number of the medications 10 in a predetermined notification. For example, the control unit 61 transmits notification information indicating that the remaining number of the medications 10 is insufficient to an apparatus of the delivery origin identified from the box ID or the like (e.g., the predetermined terminal 51) via the communication unit 65 along with patient information, the box ID, and the like.

The notification information received by the predetermined terminal 51 installed in the delivery origin is displayed on a screen, allowing the pharmacist of the delivery origin to easily know the inner box 20 in which the remaining number of the medications 10 is insufficient, the patient to which the inner box 20 has been delivered, and the remaining number of the medications 10. In this case, the pharmacist can encourage the patient to purchase the medication 10 that is insufficient in the remaining number.

As described above, in the medication management system 1 according to this embodiment, a medication tag 11 in which a specific medication ID is recorded is attached to each of a plurality of medications 10. Then, the information processing apparatus 40 transmits information including a box ID and a plurality of medication IDs that have been read at predetermined intervals to the cloud server 60. In the cloud server 60, the storage unit 62 stores the plurality of received medication IDs in association with the box ID as Not-Taken medication IDs.

In other words, a wireless tag in which a medication ID is recorded is attached to each of the one or more medications, the reading unit is configured to read the box ID and the one or more medication IDs, the first communication unit is configured to transmit information including the box ID and the one or more medication IDs that have been read by the reading unit to the cloud server, and the storage unit is configured to store the one or more medication IDs received from the first communication unit, and one or more medication statuses respectively corresponding to the one or more medication IDs, in which the one or more medication IDs are associated with the box ID, and the one or more medication statuses are stored as Not-Taken.

With this configuration, a medication ID of a medication 10 that has not been taken is stored as a Not-Taken medication ID in the cloud server 60, and a medication ID of a medication 10 that has been picked up from an inner box 20 and taken by a patient is not transmitted to the cloud server 60. This allows the cloud server 60 to deduce a medication 10 that has been taken by the patient. Note that, on the basis of reading time or receiving time of a box ID and medication IDs, it is also possible to know a time window within which the patient took a medication 10 at a level of granularity corresponding to the predetermined interval.

In addition, in the medication status management processing performed by the control unit 61, when the number of Not-Taken medication IDs stored for each box ID in the storage unit 62 is equal to or smaller than a predetermined number, predetermined notification is given.

In other words, the notification unit is further configured to give predetermined notification when a number of the one or more medication IDs corresponding to the one or more medication statuses stored as Not-Taken in the storage unit is equal to or smaller than a predetermined number.

With this configuration, when the number of Not-Taken medication IDs that are stored is equal to or smaller than the predetermined number, that is, when the number of the medications 10 available to the patient is insufficient, the above-described predetermined notification is given. Therefore, the delivery origin which has received the predetermined notification can encourage the patient to purchase the medication.

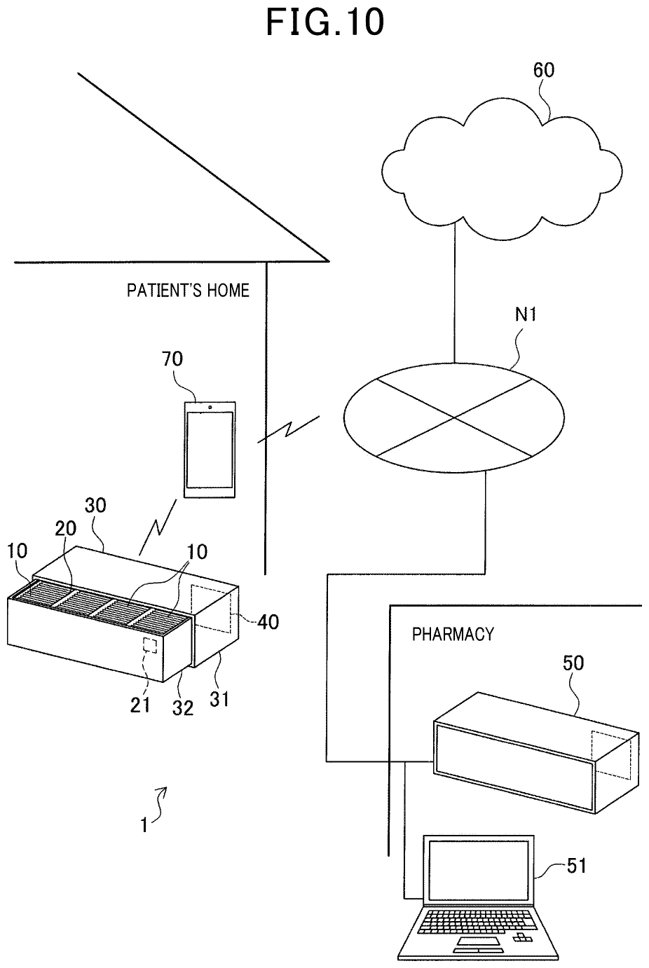

As a modification example of this embodiment, the cloud server 60 may notify a patient of at least a part of the information to be notified to the delivery origin by transmitting it to a display terminal 70 (see FIG. 10 described later) such as a smart phone managed by the patient via the network N1 (e.g., the internet or the like). For example, in a case where the number of Not-Taken medication IDs that are stored is equal to or smaller than the predetermined number as described above (YES in S113), the display terminal 70 can be used to give the predetermined notification to the patient, thereby directly prompting the patient to purchase the medication.

In this manner, in a case where the display terminal 70 that can receive notification information from the cloud server 60 and display it is used, the cloud server 60 may transmit, as information related to medications 10, for example, information indicating a time window within which the patient should take the medication to the display terminal 70. In this case, medication time information related to medication time for medications 10 is acquired from the apparatus of the delivery origin and is stored in the storage unit 62 of the cloud server 60. The medication time information is associated with at least patient information and a medication ID. On the basis of the medication time information, when it comes time for the patient to take the medication 10, the control unit 61 of the cloud server 60 transmits the information related to the medication 10 to the display terminal 70 via the communication unit 65. The display terminal 70 displays the information received from the cloud server 60 on a screen as information related to medication the patient should take.

In other words, the storage unit is configured to store medication time information related to medication time for the one or more medications, wherein the medication time information is associated with the patient information and the medication IDs, the second communication unit is configured to transmit, based on the medication time information, display information related to the one or more medications which the patient should take to a display terminal, and the display terminal is configured to display the display information received from the second communication unit.

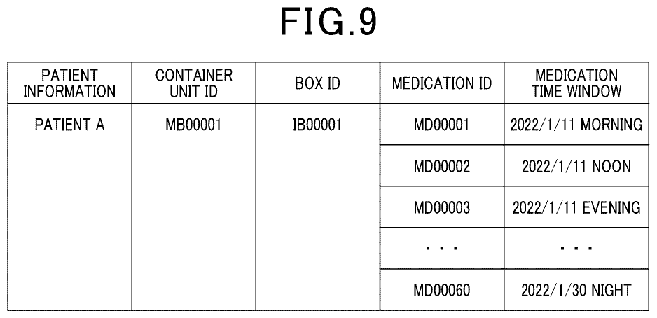

Specifically, for example, as illustrated in FIG. 9, in a case where the storage unit 62 stores, as a database in advance, patient information, medication IDs, and medication time windows for doses of medication 10 identified from the medication IDs, the control unit 61 transmits information including information related to a medication 10 identified from a medication ID of which medication time window soon comes and the medication time window to the display terminal 70. This allows the patient to easily know the medication 10 which the patient is currently supposed to take based on the information related to the medication 10 and the medication time window that are displayed on the display terminal 70. Therefore, it is possible to prevent forgetting to take a medication 10 and taking a wrong medication 10.

In a case where notification of a medication time window is given as described above, the cloud server 60 may further give notification of a container space in which a medication 10 to be taken is contained in an inner box 20. In this case, information related to a container space for a medication 10 which the patient should take is acquired from the apparatus of the delivery origin and is stored in the storage unit 62 of the cloud server 60. The information related to the container space is associated with at least a combination of a box ID and a medication ID. The information related to the container space may be further associated with patient information. Specifically, since the inside of an inner box 20 is partitioned into four compartments 22a to 22d, the cloud server 60 transmits, to the display terminal 70, information related to a medication 10 to be taken including information identifying a compartment in which the medication 10 is contained among the compartments 22a to 22d (i.e., information related to a container space) and a medication time window.

In other words, an inside of the inner box is partitioned by a divider, each of a plurality of container spaces that are partitioned corresponds to a type of the one or more medications, and the one or more medications are contained in the plurality of container spaces depending on their types, the second communication unit is configured to transmit the display information including information related to the plurality of container spaces corresponding to a type of the one or more medications which the patient should take to the display terminal, and the display terminal is configured to display the display information received from the second communication unit.

This allows the patient to easily know not only the medication 10 which the patient is currently supposed to take but also the container space for the medication 10 based on the information displayed on the display terminal 70. Therefore, it is possible to more reliably prevent taking a wrong medication 10.

In a case where the display terminal 70 is used, as illustrated in FIG. 10, the display terminal 70 instead of the information processing apparatus 40 may transmit a box ID or the like to the cloud server 60. That is, the display terminal 70 may relay communication between the information processing apparatus 40 and the cloud server 60. For example, the display terminal 70 uses Bluetooth (registered trademark), Near Field Communication (NFC), or the like to acquire information such as a box ID read by the information processing apparatus 40 using the reading unit 44. The display terminal 70 transmits the acquired information such as a box ID to the cloud server 60 at a predetermined timing. In the above-described configuration, the information processing apparatus 40 may correspond to a “reading unit” that reads a box ID from a box tag 21 of an inner box 20 contained in the container unit 30. The display terminal 70 may correspond to a “first communication unit” that acquires a box ID read by the “reading unit” and transmits information including the box ID to the cloud server 60.

In this manner, in a case where the display terminal 70 relays communication between the information processing apparatus 40 and the cloud server 60, for example, temporarily increased distance between the display terminal 70 and the information processing apparatus 40 may cause unavailability of data communication between the display terminal 70 and the information processing apparatus 40.

Thus, if the display terminal 70 falls into a state where it is unable to acquire information (e.g., medication IDs) read by the reading unit 44, the control unit 41 of the information processing apparatus 40 causes the storage unit 42 to temporarily store the medication IDs that the display terminal 70 has failed to acquire as unacquired information along with information of date and time when the medication IDs are read. Then, when the state where the medication IDs are unable to be acquired is resolved, the display terminal 70 acquires information including the unacquired information temporarily stored in the storage unit 42 and a box ID and transmits the acquired information to the cloud server 60. Note that the storage unit 42 may correspond to a “temporary storage unit”.

In other words, the reading unit includes a temporary storage unit configured to temporarily store, when the first communication unit fails to acquire the one or more medication IDs read by the reading unit, the one or more medication IDs that the first communication unit has failed to acquire as unacquired information along with reading time information, and the first communication unit is configured to transmit information including the box ID and the one or more medication IDs stored in the temporary storage unit to the cloud server.

For example, when the display terminal 70 becomes unable to communicate with the information processing apparatus 40 because the display terminal 70 is temporarily away from the information processing apparatus 40, the display terminal 70 may fall into the state where it is unable to acquire information (e.g., medication IDs) read by the reading unit 44 as described above. However, even in this case, information that has been read during this state is temporarily stored in the information processing apparatus 40 and, when the state is resolved, the display terminal 70 acquires the temporarily stored information and transmits the acquired information to the cloud server 60. Therefore, even if the relay by the display terminal 70 is temporarily interrupted, the cloud server 60 can consistently determine patient's medication statuses. In other words, it is possible to prevent loss in the medication statuses.

In addition, in a case where the display terminal 70 relays communication between the information processing apparatus 40 and the cloud server 60, the information processing apparatus 40 can authorize one preset display terminal 70 to acquire reading results and not authorize another unset mobile terminal to acquire the reading results.

In other words, the reading unit is configured to provide a reading result only to the first communication unit that is preset.

With this configuration, only a display terminal 70 which the patient owns can be permitted as a communication unit (e.g., second communication unit) to acquire reading results of the information processing apparatus 40 from an inner box 20 for the patient. In this case, since a terminal of someone other than the patient is unable to acquire the reading results, it is possible to enhance confidentiality for information such as patient's medication statuses and the like.

For example, in a case where Bluetooth communication is used between the display terminal 70 and the information processing apparatus 40, the following system configuration may be adopted. By logging in the cloud server 60, a display terminal 70, in which a dedicated application program is installed at the time of registration in the system, acquires a Bluetooth (BT) address identifying the container unit 30 and the information processing apparatus 40 and authentication information. This causes the display terminal 70 to become a master, which automatically starts connecting to the information processing apparatus 40. The display terminal 70 searches for the information processing apparatus 40 which is advertising the BT address acquired as described above and makes a connection request to the information processing apparatus 40. Then, the display terminal 70, which receives a request to transmit authentication information from the information processing apparatus 40, transmits the authentication information acquired as described above to the information processing apparatus 40 to enter a state where it can acquire reading results of the information processing apparatus 40. Meanwhile, a mobile terminal of someone else which cannot acquire the authentication information is unable to connect to the information processing apparatus 40, and is thus unable to acquire the reading results. Therefore, it is possible to enhance confidentiality for information such as patient's medication statuses and the like.

In addition, the cloud server 60 may notify a requestor which has made a predetermined request with valid privilege of at least a part of information stored in the storage unit 62. In other words, the second communication unit is configured to receive a predetermined request from a sending apparatus, and to transmit at least a part of information stored in the storage unit to the sending apparatus.

Specifically, for example, a terminal belonging to a family of the patient can receive information such as patient's medication statuses directly or indirectly from the cloud server 60 by transmitting the predetermined request to the cloud server 60. This allows the family of the patient to easily know patient's medication statuses or the like.

In the read information transmission processing performed by the information processing apparatus 40, as a timing when a box ID and a plurality of medication IDs are read, not only the timing when the set state is detected but also, for example, a timing when a predetermined time point is reached may be adopted. Moreover, a box ID and a plurality of medication IDs may be read at predetermined time intervals.

Note that the present disclosure is not limited to the above embodiments and the like and, for example, may be embodied as follows.

(1) In another embodiment of the present disclosure, the delivery origin is not limited to a pharmacy and may be, for example, a hospital or another establishment in which a medication 10 can be dispensed.

(2) In the above embodiments, the inside of an inner box 20 is partitioned into four compartments 22a to 22d as container spaces for medications 10. However, the present disclosure is not limited thereto, and the inside of an inner box 20 may be partitioned into two, three, five, or more compartments. In addition, in a case where a medication tag 11 is attached to each of medications 10, the compartments may be formed such that the orientation of a medication 10 contained in each compartment is suitable for a medication tag 11 to be read by the information processing apparatus 40.

(3) In the above embodiments, there has been described an example where the container unit 30 is a pull-out type constituted by the main unit 31 and the drawer unit 32 to contain an inner box 20, but the present disclosure is not limited thereto. The container unit 30 is a table on which an inner box 20 is placed, and the inner box 20 is placed at a predetermined position on a top surface of the container unit 30. This allows the information processing apparatus 40 of the container unit 30 to read information from a box tag 21 of the inner box 20. In this case, conceptually, placing an inner box 20 on the container unit 30 is included in storing an inner box 20 in the container unit 30.

(4) Instead of a box tag 21, an information code such as a QR code (registered trademark) in which a box ID is optically-readably recorded may be attached to a position on a surface of an inner box 20 where it can be read. In this case, an information processing terminal (e.g., display terminal 70) belonging to a patient has a function to read the information code, and an application program for the medication management system is installed therein. The information processing terminal reads a box ID from the information code by a patient's manual operation and transmits the read box ID to the cloud server 60. Similarly, instead of a medication tag 11, an information code such as a QR code in which a medication ID is optically-readably recorded may be printed on a surface of a medication 10 along with other textual information and the like. In this case, the information processing terminal reads a medication ID from the information code attached to a medication 10 taken by the patient and transmits the read medication ID and the box ID read as described above to the cloud server 60. This allows the cloud server 60 to update a medication status of the received medication ID to ‘Taken’.

1. A medication management system comprising:
a cloud server;
an inner box configured to contain one or more medications for a patient to take, wherein a wireless tag in which a box identifier, ID, is recorded is attached to the inner box;
a container unit configured to contain the inner box;
a reading unit configured to read the box ID from the wireless tag of the inner box contained in the container unit; and
a first communication unit configured to acquire the box ID read by the reading unit and to transmit information including the box ID to the cloud server;
wherein the cloud server includes:
a second communication unit configured to receive, from an apparatus of a delivery origin, the box ID and delivery time information related to a scheduled time when the inner box is to be delivered to the patient;
a storage unit configured to store the box ID, the delivery time information, and patient information related to the patient, wherein the box ID is associated with the patient information and the delivery time information is associated with the box ID;
a determination unit configured to determine whether the box ID associated with the delivery time information has been received from the first communication unit before a predetermined time has elapsed since scheduled time indicated by the delivery time information; and
a notification unit configured to give notification of information that is based on a determination result of the determination unit.

2. The medication management system according to claim 1,
wherein the first communication unit is configured to transmit information including the box ID and the patient information to the cloud server, and
the determination unit is further configured to determine whether the box ID received from the first communication unit matches the box ID that is associated, in the storage unit, with the patient information received from the first communication unit.

3. The medication management system according to claim 1,
wherein a format of the box ID is a predetermined format corresponding to information recorded in the wireless tag attached to the inner box, and
the first communication unit is configured not to transmit information that has been read by the reading unit but is not in the predetermined format to the cloud server.

4. The medication management system according to claim 1,
wherein a wireless tag in which a medication ID is recorded is attached to each of the one or more medications,
the reading unit is configured to read the box ID and the one or more medication IDs,
the first communication unit is configured to transmit information including the box ID and the one or more medication IDs that have been read by the reading unit to the cloud server, and
the storage unit is configured to store the one or more medication IDs received from the first communication unit, and one or more medication statuses respectively corresponding to the one or more medication IDs, wherein the one or more medication IDs are associated with the box ID, and the one or more medication statuses are stored as Not-Taken.

5. The medication management system according to claim 4,
wherein the notification unit is further configured to give predetermined notification when a number of the one or more medication IDs corresponding to the one or more medication statuses stored as Not-Taken in the storage unit is equal to or smaller than a predetermined number.

6. The medication management system according to claim 4,
wherein the storage unit is configured to store medication time information related to medication time for the one or more medications, wherein the medication time information is associated with the patient information and the medication IDs,
the second communication unit is configured to transmit, based on the medication time information, display information related to the one or more medications which the patient should take to a display terminal, and
the display information received from the second communication unit is displayed in the display terminal.

7. The medication management system according to claim 6,
wherein an inside of the inner box is partitioned by a divider, each of a plurality of container spaces that are partitioned corresponds to a type of the one or more medications, and the one or more medications are contained in the plurality of container spaces depending on types of the one or more medications,
the second communication unit is configured to transmit the display information including information related to the plurality of container spaces corresponding to a type of the one or more medications which the patient should take to the display terminal, and
the display information received from the second communication unit is displayed in the display terminal.

8. The medication management system according to claim 4,
wherein the reading unit includes a temporary storage unit configured to temporarily store, when the first communication unit fails to acquire the one or more medication IDs read by the reading unit, the one or more medication IDs that the first communication unit has failed to acquire as unacquired information along with reading time information, and
the first communication unit is configured to transmit information including the box ID and the one or more medication IDs stored in the temporary storage unit to the cloud server.

9. The medication management system according to claim 4,
wherein the second communication unit is configured to receive a predetermined request from a sending apparatus, and to transmit at least a part of information stored in the storage unit to the sending apparatus.

10. The medication management system according to claim 4,
wherein the reading unit is configured to provide a reading result only to the first communication unit that is preset.
